(19) United States
(12) Patent Application Publication
(10) Pub. No.: US 2024/0008891 A1
(43) Pub. Date: Jan. 11, 2024

(54) CATHETER ASSEMBLY AND USE METHOD

(71) Applicant: Terumo Kabushiki Kaisha, Tokyo (JP)

(72) Inventors: Yuya OTAKE, Shizuoka (JP); Tetsuya FUKUOKA, Kanagawa (JP)

(73) Assignee: TERUMO KABUSHIKI KAISHA, Tokyo (JP)

(21) Appl. No.: 18/339,584

(22) Filed: Jun. 22, 2023

(30) Foreign Application Priority Data
Jul. 7, 2022 (JP) 2022-109393

(51) Int. Cl. A61B 17/3207; A61M 5/00; A61M 25/00

(52) U.S. Cl. CPC A61B 17/3207; A61M 5/007; A61M 25/0097; A61M 25/0068

(57) ABSTRACT

A catheter assembly in which a microcatheter of an over-the-wire type is a first catheter and is inserted into a lumen of a guiding catheter that is a second catheter. The first and second catheters are configured to make a clearance of greater than 0.1 mm and less than or equal 0.35 mm to reduce a step, so that a distal end opening of the guiding catheter is prevented from rubbing against an inner surface of a blood vessel in a way that would otherwise rupture plaque and scatter cholesterol crystals.

### CROSS-REFERENCES TO RELATED APPLICATIONS

This application claims priority to Japanese Patent Application No. 2022-109393 filed on Jul. 7, 2022, the entire content of which is incorporated herein by reference.

### TECHNOLOGICAL FIELD

The present disclosure generally relates to a catheter assembly used by being inserted into a heart or a peripheral tissue of the heart, in particular, left and right coronary arteries (hereinafter, also simply referred to as “coronary arteries”). In particular, the disclosure relates to a method for using the catheter assembly for preventing cholesterol embolisms, which may rarely occur when advancing, into a blood vessel, a guiding catheter used for introducing a treatment catheter such as an inflation catheter for percutaneous transluminal coronary angioplasty (PTCA) or a stent delivery catheter into a target site of the blood vessel.

### BACKGROUND DISCUSSION

Cholesterol, which is a fat-soluble compound dissociated from low-density lipoprotein (LDL) and crystallized, may be accumulated in intravascular atheroma or blood vessel wall plaque caused by arteriosclerosis.

Cholesterol crystal embolism (CCE) refers to occlusion or an inflammatory response occurring in a fine blood vessel such as a peripheral blood vessel when plaque in a blood vessel is ruptured, for example, due to catheter treatment or thrombolytic therapy and needle-shaped cholesterol crystals having a diameter of 55 μm to 200 μm diffuse into a body as disclosed in Hase, The Journal of the Japanese Society of Internal Medicine, 105(5) 2016: 850-856.

It has been said that BlueToe syndrome due to occlusion of the peripheral artery by CCE (in Hase, The Journal of the Japanese Society of Internal Medicine, 105(5) 2016: 850-856), and acute renal failure that occludes glomeruli (in Vuurmans, Heart, 2010; 96: 1538-1542) are less likely to occur in transradial artery intervention (TRI) than in transfemoral intervention (TFI) in percutaneous coronary intervention (PCI).

Japanese Patent Application Publication No. 2020-151465 A discloses a method for suctioning and removing cholesterol crystals and the like at a lesion, but it is not intended to suppress scattering of cholesterol crystals generated when the guiding catheter is advanced to the lesion. Takahashi et al., The American Journal of Cardiology, Jan. 2, 2022 (online ahead of print) discloses that a catheter referred to as an over-the-wire type inner sheath is inserted into a guiding catheter, and U.S. Pat. No. 10,322,260 and U.S. Patent Application Publication No. 2007/0149927 disclose a catheter assembly of a guiding catheter inserted from the radial artery and an inner sheath, a clearance being a difference between an inner diameter of a guiding catheter distal end opening and an outer diameter of the inner sheath is 0.1 mm, and in a case where the guiding catheter and the inner sheath are longer than 2 m, friction might increase.

An insertion assisting tool of a rapid exchange type as in U.S. Patent Application Publication No. 2009/0264865 have relatively no clearance between the tool and an inner diameter of a guiding catheter or a clearance of less than 0.1 mm, there is a possibility that the tool does not protrude from a guiding catheter distal end opening.

### SUMMARY

A catheter assembly comprising: a first catheter comprised of a first shaft that includes a distal end and a proximal end at opposite axial ends of the first shaft, the first shaft including a first distal end opening at the distal end of the first shaft and a first proximal end opening at the proximal end of the first shaft, the first catheter including a first lumen extending between the first distal end opening and the first proximal end opening; a second catheter comprised of a second shaft that includes a distal end and a proximal end at opposite axial ends of the second shaft, the second shaft including a second distal end opening at the distal end of the second shaft and a second proximal end opening at the proximal end of the second shaft, the second catheter including a second lumen extending between the second distal end opening and the second proximal end opening, the second catheter including a second hub at the proximal end of the second shaft and communicating with the second lumen, the second catheter having both an outer diameter and an inner diameter at the second distal end opening; the first catheter being positionable in the second lumen of the second shaft and being axially positionable relative to the second shaft such that a distal portion of the first shaft extends distally beyond the distal end of the second shaft; and a clearance between an inner surface of the second shaft and an outer surface of the first shaft at the second distal end opening when the first catheter is positioned in the second lumen of the second shaft and the distal portion of the first shaft extends distally beyond the distal end of the second shaft, and wherein the clearance is greater than 0.1 mm and less than or equal to 0.35 mm.

A catheter assembly comprising: a first catheter comprised of a first shaft that includes a first distal end opening, a first proximal end opening and a first lumen extending between the first distal end opening and the first proximal end opening; a second catheter comprised of a second shaft that includes a second distal end opening, a second proximal end opening and a second lumen extending between the second distal end opening and the second proximal end opening, the second catheter including a hub at a proximal end of the second shaft, the second catheter having both an outer diameter and an inner diameter at the second distal end opening, the first catheter being positioned in the second lumen of the second shaft, a guide wire located in the first lumen in the first shaft; and a clearance between an inner surface of the second shaft and an outer surface of the first shaft at the second distal end opening is greater than 0.1 mm and less than or equal to 0.35 mm.

In a method of use of a catheter assembly for reducing cholesterol crystal embolism occurring during catheter treatment, the method comprising: positioning the catheter assembly for insertion into a first blood vessel, the catheter assembly comprising: a first catheter comprised of a first shaft that includes a first distal end opening, a first proximal end opening and a first lumen extending between the first distal end opening and the first proximal end opening; a second catheter comprised of a second shaft that includes a second distal end opening, a second proximal end opening and a second lumen extending between the second distal end opening and the second proximal end opening, the second catheter including a hub at a proximal end of the second shaft, the second catheter having both an outer diameter and an inner diameter at the second distal end opening; the first catheter being positioned in the second lumen of the second shaft; a guide wire located in the first lumen in the first shaft; and a clearance between an inner surface of the second shaft and an outer surface of the first shaft at the second distal end opening, the clearance being greater than 0.1 mm and less than or equal to 0.35 mm; inserting the catheter assembly into the first blood vessel; advancing the catheter assembly along the first blood vessel while a distal portion of the first shaft extends distally beyond the distal end of the second shaft and while the guide wire is located in the first lumen in the first shaft and extends distally beyond the distal end of the first shaft; advancing the catheter assembly from the first blood vessel to a second blood vessel having a branch that includes a third blood vessel; and advancing at least the second catheter into the third blood vessel having a lesion on which catheter treatment is to be performed.

According to the disclosure here, to eliminate a large step, which is a clearance of about 1 mm between a distal end opening of a guiding catheter and a guide wire inserted thereinto, a catheter assembly in which a microcatheter of an over-the-wire type is inserted into a lumen of the guiding catheter is used to make the clearance greater than 0.1 mm and less than or equal to 0.35 mm to reduce the step, so that the distal end opening of the guiding catheter is prevented from rubbing an inner surface of a blood vessel to rupture plaque and scatter cholesterol crystals. In the absence of the microcatheter, a relatively large clearance would exist between the inner surface of the guiding catheter and the outer surface of the guide wire, and so the guiding catheter would be able to move laterally a relatively large amount, meaning the guiding catheter may rub against the inner surface of the blood vessel. Positioning the catheter (microcatheter) inside the guiding catheter reduces the clearance between the inner surface of the guiding catheter and the outer surface of the microcatheter and so the guiding catheter is not able to move laterally to the same extent, thus reducing the likelihood that the guiding catheter will rub against the inner surface of the blood vessel. If the clearance becomes too small, frictional resistance between the inner surface of the guiding catheter and the outer surface of the microcatheter may undesirably increase. The catheter assembly thus seeks to identify a clearance that will both lessen the possibility that the guiding catheter will rub against the inner surface of the blood vessel and reduce frictional resistance between the inner surface of the guiding catheter and the outer surface of the microcatheter.

### BRIEF DESCRIPTION OF THE DRAWINGS

FIG. 1 is a cross-sectional view in an axial direction of a catheter assembly according to an embodiment.

FIG. 2 is an enlarged view of a distal end of the catheter assembly according to the embodiment.

FIGS. 3A, 3B, and 3C are cross-sectional views taken along the section lines 3A-3A, 3B-3B, and 3C-3C, respectively, of the catheter assembly according to the embodiment.

FIG. 4 is an enlarged cross-sectional view of the catheter assembly shown in FIG. 3B, illustrating dimensions of the catheter assembly.

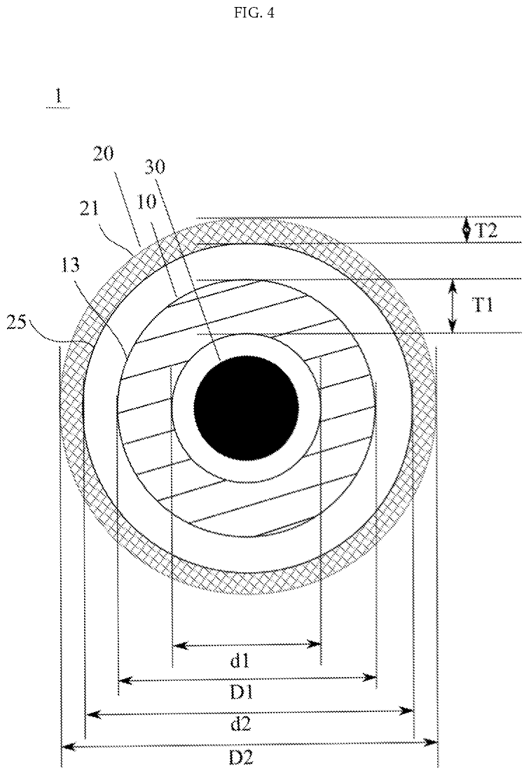

FIG. 5 is Table 1, which illustrates different embodiments of the catheter assembly.

### DETAILED DESCRIPTION

Set forth below with reference to the accompanying drawings is a detailed description of embodiments of a catheter assembly and a method for using the catheter assembly.

Here, a first catheter is referred to as, for example, a microcatheter 10. The microcatheter 10 can include a first shaft 11 that is a microcatheter shaft, a first distal opening portion 18 that is a distal opening portion of a microcatheter of the microcatheter shaft 11, a first proximal opening portion 19 that is a proximal opening portion of the microcatheter, and a first lumen 15 that is a microcatheter lumen communicating with the first distal opening portion 18 and the first proximal opening portion 19.

The first lumen 15 of the first shaft 11 is intended to allow only a guidewire 30 to pass through the first lumen 15 of the first shaft 11, and does not allow a treatment catheter such as a general balloon catheter or a stent delivery catheter to pass through the first lumen 15 of the first shaft 11. Alternatively, the first lumen 15 may also allow a treatment catheter such as a balloon catheter or a stent delivery catheter having a reduced outer diameter to pass through the first lumen 15 of the first shaft 15.

The first shaft 11 may have a tapered distal end or may be embedded with a reinforcing body. The reinforcing body may be embedded in at least a base portion 12 of the first shaft 15 at a position on a distal side with respect to the first proximal opening portion 19. The reinforcing body may be, for example, a braiding or coil-winding a metal wire such as a stainless alloy, a nickel-titanium alloy, or tungsten.

Here, a second catheter is referred to as a guiding catheter 20 having a distal tip used for introducing the treatment catheter such as a dilation catheter for PTCA or a stent delivery catheter into a target site of a blood vessel, the guiding catheter 20 can include a distal tip 21, a second shaft 22 that is a shaft of a guiding catheter 20, a second distal opening portion 25 that is a distal opening portion of the guiding catheter, a second proximal opening portion 26 that is a proximal opening portion of the guiding catheter, and a second lumen 23 that is a lumen of the guiding catheter communicating with the second distal opening portion 25 and the second proximal opening portion 26, and a hub (second hub) 24 of the guiding catheter, communicating with the second distal opening portion 25 and the second proximal opening portion 26, is attached to a proximal end of the second shaft 22.

The second shaft 22 may further include the distal tip 21 at a distal end of the second shaft 22. The second shaft 22 may be obtained or formed, for example, by winding a reinforcing body around a PTFE inner layer, and further providing an outer layer outside of the reinforcing body so that the wound reinforcing body is embedded. The reinforcing body may be embedded in the second shaft 22 at least from a position closer to the proximal end than the distal tip 21 to the second proximal end opening 26. The reinforcing body may be formed, for example, by braiding or coil-winding a metal wire such as a stainless alloy, a nickel-titanium alloy, or tungsten. A hydrophilic lubricating coat layer may be provided on a part of an outer surface of each of the distal tip 21 and the second shaft 22.

The second catheter 20 may have a distal shape having a curved or bent portion at the distal end of the second catheter 20, such as a Judkins shape, but such a distal shape is not necessarily required.

A hub (first hub) 17 of the microcatheter, communicating with the first distal opening portion 18 and the first proximal opening portion 19, is attached to a proximal end of the first shaft 11. The first proximal opening portion 19 and the first lumen 15 are communicating with a first hub lumen. The first shaft 11 may include a lock adapter 16 for fixation on a first hub 17 at a proximal end.

In order to reduce the cost, the first shaft 11 may be a shaft which a single-layer tube may have no reinforcing body, a shaft to which single-layer tubes having different hardness are connected, or a shaft that is a multilayer tube having a reinforcing body or an angiographic catheter having a reinforcing body, an inner layer, and an outer layer, which includes a physical property transition portion that is hardened from the distal end toward the base portion or has a hydrophilic lubricating coat on the distal end.

The first catheter 10 is inserted into the second lumen 23 of the second catheter 20 and advanced to a predetermined position to achieve a state in which the first shaft 11 of the first catheter 10 protrudes from the guiding catheter distal end opening 25. The predetermined position is not especially limited, but when the first catheter 10 advances to the predetermined position, the guide wire 30 and the first distal end opening 18, preferably the distal portion of the first shaft 11, protrude from (i.e., distally beyond) the second distal end opening 25 to the distal end side. A distal end protruding length of the first shaft is a distance between the first distal end opening 18 and the second distal end opening 25, which is, for example, 10 mm to 200 mm, preferably 30 mm to 150 mm.

A length from the first distal end opening 18 to the first proximal end opening 19 is defined as a first shaft length L1, and the first shaft length L1 may be, for example, a length from the first distal end opening 18 to a first shaft proximal end or a length from the first distal end opening 18 to the first proximal end opening 19, an outer diameter of the first shaft 10 at a position of the distal end opening of the second catheter 20 is defined as a first outer diameter D1, an inner diameter of the first catheter 10 is defined as a first inner diameter d1, a first wall thickness of the first catheter 10 is defined as T1, a length from the second distal end opening 25 to the second proximal end opening 26 is defined as a second shaft length L2, an outer diameter of the distal end opening of the second catheter 20 is defined as a second outer diameter D2, an inner diameter of the distal end opening of the second catheter is defined as a second inner diameter d2, and a difference between the second inner diameter d2 and the first outer diameter D1 is defined as a clearance CL.

Although the first wall thickness T1 of the first catheter 10 is defined as (D1−d1)/2 and a second wall thickness T2 of the second catheter 20 is defined as (D2−d2)/2, an average value of measured wall thicknesses may be used.

Each dimension may be constant from the distal end to the proximal end, D2 may be smaller than an outer diameter of the second proximal opening 26, or d2 may be smaller than an inner diameter of the proximal opening portion 26.

As for the clearance CL, in order to eliminate, for example, a large step which is a clearance of about 1 mm between the second distal end opening 25 of the second catheter 20 and the guide wire 30 inserted into the second distal end opening 25 of the second catheter 20, a catheter assembly 1 obtained by inserting or positioning the first catheter 10 of an over-the-wire type into the second lumen 23 of the second catheter 20 can be used to make the clearance the CL about 0.1 mm<CL≤0.35 mm to reduce the step, so that the second distal end opening 25 or the distal tip 21 is prevented from rubbing an inner surface of the blood vessel to rupture plaque and scatter a cholesterol crystal.

The clearance CL is not particularly limited as long as the clearance CL is a step smaller than the difference between the inner diameter of the second catheter and the outer diameter of the guidewire, is preferably 0.1 mm<CL≤0.35 mm, and is more preferably 0.15 mm CL 0.3 mm. Accordingly, frictional resistance between an inner surface of the second lumen 23 and an outer surface of the first shaft 11 can be reduced.

As the guide wire 30, an angiographic guide wire having an outer diameter of 0.89 mm (0.035 inch) referred to as an angiographic guide wire (for example, a Radifocus Guide Wire M® manufactured by Terumo Corporation, hereinafter, simply referred to as a guide wire) is mainly used, but a therapeutic guide wire used for PTCA or the like referred to as a PTCA guide wire (for example, a Terumo PTCA guide wire, hereinafter referred to as a therapeutic guide wire) having an outer diameter of 0.36 mm (0.014 inch) may also be used. The outer diameter or use of the guide wire 30 is not especially limited.

Hereinafter, an operation (procedure) when engaging with the left coronary artery ostium having a lesion using the guiding catheter 20, which is the second catheter, and the catheter assembly 1 is described in detail. These operations are performed while checking a position and a posture of the catheter assembly 1 under X-ray fluoroscopy.

Here, the right radial artery into which the catheter assembly 1 is inserted is a first blood vessel, the aorta is a second blood vessel with a branch, and the left coronary artery is a third blood vessel (branch of the second blood vessel) having a lesion on which catheter treatment is performed.

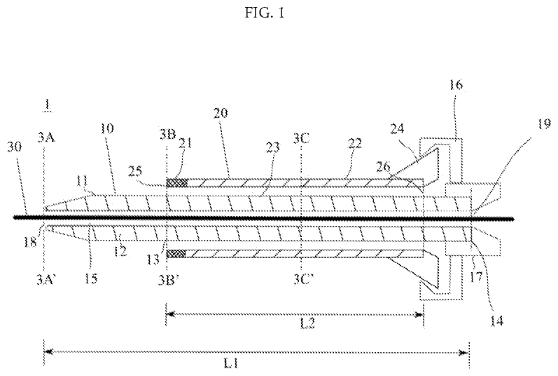

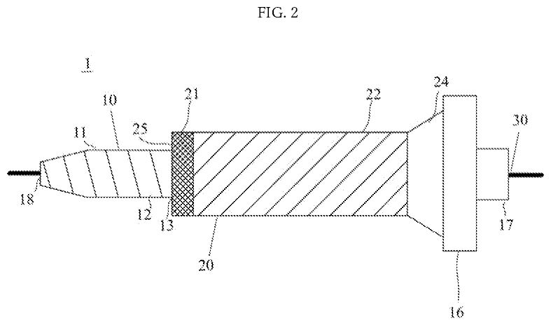

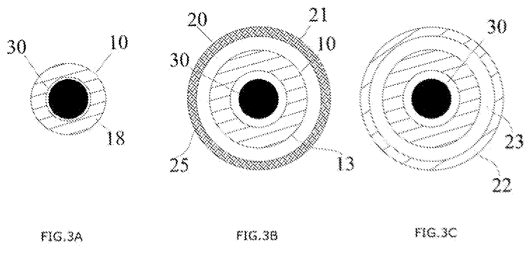

The right radial artery is punctured with a catheter introducer, and the catheter assembly 1 is inserted into the catheter introducer in a state in which the guide wire 30 having an outer diameter of 0.89 mm (0.035 inch) is inserted into (positioned in) the first lumen 15 of the microcatheter 10, which is the first catheter of the catheter assembly 1, as illustrated in FIGS. 1-3. In a state in which both the guide wire 30 and the microcatheter 10 protrude from the second distal end opening of the guiding catheter 20, a distal shape of the guiding catheter 10 is substantially linear due to rigidity of the guide wire 30.

After the catheter assembly 1 is inserted into the right radial artery from the catheter introducer in this manner, a distal end of the catheter assembly is advanced from the brachiocephalic artery to the ascending aorta with the guide wire preceding the same (i.e., the guide wire is advanced to the ascending aorta after the catheter assembly). When the distal tip 21 of the guiding catheter 20 is located about 10 cm above the left coronary artery ostium of the left coronary artery or exceeds the aortic arch, the advance of the catheter assembly 1 is stopped, and the microcatheter 10 and the guide wire 30 are removed to make the distal shape of the guiding catheter its original curved shape (natural state). That is, after removing the microcatheter and the guide wire 30 from the guiding catheter 10, the distal shape or distal portion of the guiding catheter 20 assumes its original curved shape.

Next, when the guiding catheter 20 is slowly pushed forward while checking a distal end position of the guiding catheter 20 or the position of the distal tip 21, the distal tip 21 of the guiding catheter 20 moves downward while being in contact with a left inner wall of the ascending aorta and is inserted into the left coronary artery ostium. The distal shape of the guiding catheter 20 at that time is a shape that facilitates engagement, for example, with the left coronary artery ostium.

In a case where the distal end of the guiding catheter 20 is directed in a direction different from the left coronary artery ostium, the guiding catheter 20 is slightly rotated in a counterclockwise direction to direct the distal end in the direction of the left coronary artery ostium, and is slowly pushed forward in the direction of the left coronary artery ostium. As a result, the distal end opening can be rather easily inserted into the left coronary artery ostium and engaged with the left coronary artery ostium in this state.

Next, a connector of a contrast agent injection instrument is connected to the second hub 24 including a Luer taper of the guiding catheter 20, and a contrast agent is injected. The injected contrast agent passes through the second lumen 23 via the second hub 24, and is ejected from the second distal end opening 25 into the left coronary artery. As a result, a stenosed part (diseased part) in the left coronary artery can be imaged and a position of the stenosed part (diseased part) in the left coronary artery can be identified, but this operation is not essential.

Next, the connector of the contrast agent injection instrument is removed from (disconnected from) the second hub 24. Thereafter, a treatment catheter such as a balloon catheter for PTCA can be inserted into the second lumen 23 from the second hub 24 together with a new therapeutic guide wire having an outer diameter of 0.36 mm (0.014 inch) to reach the stenosed part as a target site and treatment can be performed on the stenosed part.

Specifically, a balloon portion of the balloon catheter is pushed forward to the stenosed part with the therapeutic guide wire preceding the same, and the balloon is dilated or expanded to perform a dilation treatment of the stenosed part.

The guiding catheter 20 may be engaged with the coronary artery using the catheter assembly 1 disclosed here via the left radial artery, or may be inserted from the femoral artery via the abdominal aorta. In these cases, via the left radial artery or the femoral artery via the abdominal aorta, the procedure similar to that described above can be performed.

In the above description, the case where the balloon catheter for PTCA is used as the treatment catheter is described, but the treatment catheter is not limited to the balloon catheter, for example, for PTCA. For example, in a case where a stent delivery catheter that delivers a stent to release at a stenosed part is used, for example, the procedure is substantially similar.

Also in a case where there is a stenosed part in the right coronary artery and this is subjected to dilation treatment, the procedure similar to that described above can be performed according to this disclosure.

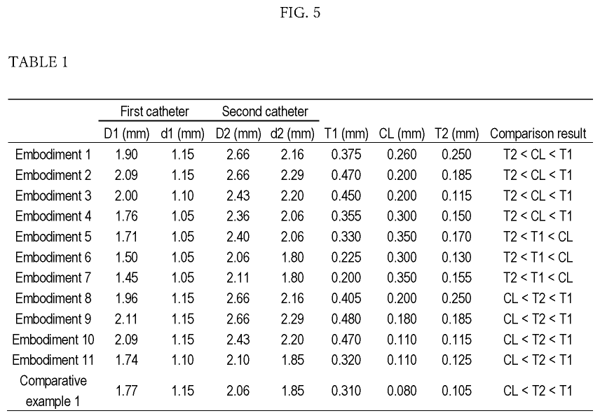

All of the embodiments of Table 1 (FIG. 5) satisfy 0.1 mm<CL≤0.35 mm, or the guiding catheter 20 having an outer diameter of 2.33 mm (7 Fr), an outer diameter of 2.66 mm (8 Fr) or more as in Embodiments 1 to 4 may have a combination satisfying T2<CL<T1. An increase in friction between the inner surface of the second lumen 23 of the guiding catheter 20 and the outer surface of the first shaft 11 is prevented. It is possible to reduce CCE said to be more likely to occur especially when used in a Femoral approach than in a Radial approach.

Alternatively, a combination of T2<T1<CL may be adopted as in Embodiments 5 to 7. Since the CL is relatively large, it is possible to help prevent the friction between the inner surface of the second lumen 23 of the guiding catheter 20 and the outer surface of the first shaft 11 from increasing, and to prevent the CCE with an occurrence frequency lower than that in the insertion from the femoral artery when the guiding catheter 20 is inserted from the radial artery.

Furthermore, when the distal tip 21 and the second shaft are relatively thin as in Embodiments 8 to 11, specifically, when the guiding catheter 20 having a small T2 is used, CL<T2<T1 is set, and it is possible to reduce an outer diameter of the guiding catheter 20 while preventing the thin second distal opening portion 25 or the distal tip 21 from crushing the plaque on a blood vessel surface. For example, since the guiding catheter 20 is used for cases of treating a cerebrovascular and treating a coronary artery, an abdominal artery, a lower limb artery, or the like by being inserted from a radial artery, it is particularly preferable to increase an inner diameter and reduce an outer diameter.

The inner diameters and the outer diameters of the distal opening portion and the shaft may be constant or may decrease toward the distal end.

An application of the catheter assembly 1 disclosed as an example of the new catheter assembly disclosed here is not especially limited, and can be applied to, for example, the guiding catheter 20 used for introducing an atherectomy catheter and an ultrasound catheter in addition to the above-described catheter. In addition, it is needless to say that a site from which the guiding catheter 20 is to be inserted into a living body is not limited to the radial artery, and may be the femoral artery. It is needless to say that the lesion area to be treated is not limited to the coronary artery, and may be the lower limb artery, a blood vessel connected to an organ such as a liver, a prostate, or a uterine artery, a blood vessel leading to a cerebrovascular such as a neck artery or a vertebral bra artery, the cerebrovascular, or the like.

Alternatively, the guiding catheter 20 may be packaged with the microcatheter 10, the packaged and sterilized single guiding catheter 20 and single microcatheter 10 may be assembled at the time of the catheter treatment and used as the catheter assembly 1, or the guiding catheter 20 and the microcatheter 10 may be packaged and sterilized in an assembled state and immediately taken out and used in an operation room.

The detailed description above describes embodiments of a catheter assembly and method of use representing examples of the new catheter assembly and method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A catheter assembly comprising:
a first catheter comprised of a first shaft that includes a distal end and a proximal end at opposite axial ends of the first shaft, the first shaft including a first distal end opening at the distal end of the first shaft and a first proximal end opening at the proximal end of the first shaft, the first catheter including a first lumen extending between the first distal end opening and the first proximal end opening;
a second catheter comprised of a second shaft that includes a distal end and a proximal end at opposite axial ends of the second shaft, the second shaft including a second distal end opening at the distal end of the second shaft and a second proximal end opening at the proximal end of the second shaft, the second catheter including a second lumen extending between the second distal end opening and the second proximal end opening, the second catheter including a second hub at the proximal end of the second shaft and communicating with the second lumen, the second catheter having both an outer diameter and an inner diameter at the second distal end opening;
the first catheter configured to be positioned in the second lumen of the second shaft and to be axially positionable relative to the second shaft such that a distal portion of the first shaft extends distally beyond the distal end of the second shaft; and
a clearance between an inner surface of the second shaft and an outer surface of the first shaft at the second distal end opening when the first catheter is positioned in the second lumen of the second shaft and the distal portion of the first shaft extends distally beyond the distal end of the second shaft, the clearance being greater than 0.1 mm and less than or equal to 0.35 mm.

2. The catheter assembly according to claim 1, wherein the first shaft has a tapered distal end.

3. The catheter assembly according to claim 1, wherein the second catheter is a guiding catheter having a distal tip configured to introduce a treatment catheter.

4. The catheter assembly according to claim 1, wherein the distal end of the second shaft includes a curved or bent portion.

5. The catheter assembly according to claim 4, wherein the distal end of the second shaft has a Judkins shape.

6. The catheter assembly according to claim 1, wherein a distance between the first distal end opening and the second distal end opening is 10 mm to 200 mm.

7. The catheter assembly according to claim 1, wherein the clearance is greater than or equal to 0.15 mm and less than or equal to 0.3 mm.

8. The catheter assembly accordingly to claim 1, further comprising:
a guide wire configured to be received in the first lumen in the first shaft.

9. The catheter assembly according to claim 8, wherein guide wire is an angiographic guide wire having an outer diameter of 0.89 mm.

10. The catheter assembly according to claim 9, wherein guide wire is a therapeutic guide wire having an outer diameter of 0.36 mmm.

11. A catheter assembly comprising:
a first catheter comprised of a first shaft that includes a first distal end opening, a first proximal end opening and a first lumen extending between the first distal end opening and the first proximal end opening;
a second catheter comprised of a second shaft that includes a second distal end opening, a second proximal end opening and a second lumen extending between the second distal end opening and the second proximal end opening, the second catheter including a hub at a proximal end of the second shaft, the second catheter having both an outer diameter and an inner diameter at the second distal end opening, the first catheter being positioned in the second lumen of the second shaft, a guide wire located in the first lumen in the first shaft; and
a clearance between an inner surface of the second shaft and an outer surface of the first shaft at the second distal end opening is greater than 0.1 mm and less than or equal to 0.35 mm.

12. The catheter assembly according to claim 11, wherein the first shaft has a tapered distal end.

13. The catheter assembly according to claim 11, wherein the second catheter is a guiding catheter having a distal tip configured to introduce a treatment catheter.

14. The catheter assembly according to claim 11, wherein the distal end of the second shaft has a Judkins shape.

15. The catheter assembly according to claim 11, wherein a distance between the first distal end opening and the second distal end opening is 10 mm to 200 mm, and the clearance is greater than or equal to 0.15 mm and less than or equal to 0.3 mm.

16. The catheter assembly accordingly to claim 11, further comprising:
a guide wire configured to be received in the first lumen in the first shaft.

17. A method of use of a catheter assembly for reducing cholesterol crystal embolism occurring during catheter treatment, the method comprising:
positioning the catheter assembly for insertion into a first blood vessel, the catheter assembly including a first catheter comprised of a first shaft that includes a first distal end opening, a first proximal end opening and a first lumen extending between the first distal end opening and the first proximal end opening, a second catheter comprised of a second shaft that includes a second distal end opening, a second proximal end opening and a second lumen extending between the second distal end opening and the second proximal end opening, the second catheter including a hub at a proximal end of the second shaft, the second catheter having both an outer diameter and an inner diameter at the second distal end opening, the first catheter being positioned in the second lumen of the second shaft, a guide wire located in the first lumen in the first shaft, and a clearance between an inner surface of the second shaft and an outer surface of the first shaft at the second distal end opening is greater than 0.1 mm and less than or equal to 0.35 mm;
inserting the catheter assembly into the first blood vessel;
advancing the catheter assembly along the first blood vessel while a distal portion of the first shaft extends distally beyond the distal end of the second shaft and while the guide wire is located in the first lumen in the first shaft and extends distally beyond the distal end of the first shaft;
advancing the catheter assembly from the first blood vessel to a second blood vessel having a branch that includes a third blood vessel; and
advancing at least the second catheter into the third blood vessel having a lesion on which catheter treatment is to be performed.

18. The method according to claim 17, further comprising:
removing the first catheter and the guide wire to allow a distal shape of the second catheter to return to a natural state;

19. The method according to claim 17, further comprising:
injecting a contrast agent into the second lumen via the hub into the third blood vessel having the lesion; and
imaging the lesion in the third blood vessel on which the catheter treatment is to be performed.

20. The method according to claim 17, further comprising:
advancing a treatment catheter into the second lumen together with a therapeutic guide wire to the lesion on which catheter treatment is to be performed; and
treating the lesion with the treatment catheter.
